(19) United States
(12) Patent Application Publication
(10) Pub. No.: US 2026/0315244 A1
(43) Pub. Date: Oct. 15, 2026

(54) SPINAL IMPLANT SYSTEM FOR A SPINAL SPACER

(71) Applicant: COUSIN BIOTECH, WERVICQ SUD (FR)

(72) Inventors: Eléonore HOFFMANN, LINSELLES (FR); Michel CAILLIBOTTE, LILLE (FR)

(21) Appl. No.: 19/487,483

(22) PCT Filed: Mar. 26, 2024
(86) PCT No.: PCT/EP2024/058052

§ 371 Date: Nov. 25, 2025

(30) Foreign Application Priority Data
Jun. 1, 2023 (FR) 2305473

(51) Int. Cl. A61F 2/46; A61B 17/00; A61B 17/70; A61F 2/44

(52) U.S. Cl. CPC A61F 2/4611; A61B 17/7061; A61B 17/7065; A61F 2/44; A61B 2017/00367; A61B 2017/00477; A61F 2002/4615; A61F 2002/4627; A61F 2002/4635

(57) ABSTRACT

A spinal implant system for a spinal spacer that is at least partially elastically deformable between a deployed state and a compressed state, the implant system comprising a compression device configured to cause said spinal spacer to switch from the deployed state to the compressed state; a guide device having an open distal end and comprising an implantation tube, the guide device being able to be connected to said compression device; and a pusher device configured to cooperate with the compression device, when the guide device is connected thereto, to push the spinal spacer in the compressed state from the cavity of the compression device into the implantation tube of the guide device; and with the guide device to push the spinal spacer in the compressed state along the implantation tube up to the distal end of the guide device, so as to extract said spinal spacer out of the guide device.

### CROSS REFERENCE TO RELATED APPLICATIONS

This application is the U.S. National Stage, under 35 U.S.C. § 371, of International Application No. PCT/EP2024/058052, filed Mar. 26, 2024, which claims the benefit of FR Patent Application no. 2305473 filed on Jun. 1, 2023, the contents of which are hereby incorporated by reference herein.

### TECHNICAL FIELD

The present disclosure relates to the technical field of systems for implanting a spinal spacer between two vertebrae. Such a spinal spacer, which may also be referred to as a spacer device or an interspinous spacer, helps maintain normal spacing between two adjacent vertebrae between which it is arranged. More precisely, the spinal spacer may be positioned between the spinous processes or between the laminae of two adjacent vertebrae. One advantage is that this helps combat degeneration of the intervertebral discs, which may cause pain, numbness, weakness or even paralysis in various parts of the body.

This spinal spacer provides treatment by restoring the intervertebral spacing, allowing loads to be distributed between the vertebrae.

The present disclosure relates more specifically to systems for implanting spinal spacers that are elastically deformable and therefore flexible. Implanting spinal spacers of this type eliminates the need to dissect muscle tissue and the supra- and interspinous ligaments, which is necessary when implanting hard or rigid spacers that are not elastically deformable. In particular, they are less invasive to implant than rigid spinal spacers.

Compared to rigid spacers, elastically deformable spinal spacers also improve the mobility and flexibility of the vertebral column, allowing more natural movements similar to those of a healthy vertebral column.

### PRIOR ART

Spinal implant systems for elastically deformable spinal spacers are known, for example that described in document ITFI20030292A1. The system described in this document comprises a pipe with a square cross section configured to receive a spinal spacer in a compressed state, via an opening formed at the proximal end of the pipe. The spacer is then moved along the pipe, by means of a rod, until it exits the pipe so that it can be implanted between the spinous processes of a patient.

Firstly, although it is not described in this document, putting the spinal spacer in the compressed state is not a straightforward operation and requires a suitable tool which is not described in this document. Indeed, the system described in this document cannot be used to place the spinal spacer in a compressed state, meaning that the operator must him-or herself compress the spinal spacer by means of a separate tool, for example pliers. This step of compressing the spinal spacer requires considerable force. Furthermore, there is a risk of the spinal spacer accidentally returning to the deployed state, which would mean having to compress the spinal spacer again, could cause it to fall, or could indeed injure the operator.

Moreover, positioning the compressed spinal spacer in the pipe is particularly complicated. In order to position the spacer inside the pipe, it needs to be kept in the compressed state while it is being inserted via the proximal opening of the pipe, which requires a great deal of precision and is very complicated. In particular, complex handling operations are required in order to prevent the spinal spacer from being accidentally deployed while it is being inserted through the opening of the pipe.

Furthermore, the system described in this document implants the spacer via a cannula with a large cross section, which is not suitable for implementing a minimally invasive implantation technique (minimally invasive spine surgery or MISS). Indeed, such a minimally invasive technique requires the use of a pipe or trocar with a small diameter, generally less than 16 millimeters, through which the spacer can be passed and transported to between the vertebrae. According to this minimally invasive technique, the trocar is used to guide the spacer up to the area to be treated so that the spacer does not damage tissues when it is being implanted.

Neither the system described in this prior art document nor any other known prior art document provides a solution that can be used to arrange a spinal spacer in a trocar with a small diameter suitable for minimally invasive implantation. As a result, the minimally invasive technique is not yet used to implant spinal spacers.

### DISCLOSURE OF THE INVENTION

One aim of the present disclosure is to propose a spinal implant system for a spinal spacer that overcomes the disadvantages mentioned above.

For this purpose, the disclosure relates to a spinal implant system for a spinal spacer that is at least partially elastically deformable between a deployed state and a compressed state, the implant system comprising:
a compression device having a cavity intended to receive the spinal spacer in the deployed state, the compression device being configured to exert a compression force on said spinal spacer situated in said cavity so as to cause said spinal spacer to switch from the deployed state to the compressed state;
a guide device having an open distal end and comprising an implantation tube, the guide device being able to be connected to said compression device; and
a pusher device configured to cooperate:
   with the compression device, when the guide device is connected thereto, to push the spinal spacer in the compressed state from the cavity of the compression device into the implantation tube of the guide device; and
with the guide device, to push the spinal spacer in the compressed state along the implantation tube up to the distal end of the guide device, so as to extract said spinal spacer out of the guide device.

The deployed state of the spinal spacer corresponds to a resting state in which it is not compressed. Compressing the spinal spacer helps to reduce its space requirement so that it can be inserted between two vertebrae. The compressed state corresponds an insertion state of the spinal spacer. In the compressed state, the spinal spacer has a reduced space requirement in at least one direction, preferably a reduced height, facilitating its insertion into the guide device in particular.

“Compression” should be understood to mean a rectilinear pressure force exerted on the spacer. This compression advantageously flattens the spacer.

The spinal spacer is flexible. It tends to return to the deployed state when it is compressed. It also tends to return to the deployed state when it is positioned between two vertebrae.

The spinal implant system according to the disclosure is particularly suitable for implanting an elastically deformable spinal spacer such as that described in EP 4 149 376 A1 or in U.S. Pat. No. 8,118,839 B2. The spinal spacer advantageously has at least one, and preferably two, elastically deformable wings.

Advantageously, the spinal implant system is suitable for performing the spinal implantation of a spinal spacer such as that described in EP 4 149 376 A1, the description of which is incorporated into the present text.

The spinal spacer advantageously extends in a main direction. It is advantageously deformable in at least one direction transverse to its main direction.

In a non-limiting manner, the cavity of the compression device may be cylindrical. The cavity advantageously has a longitudinal direction.

The compression device advantageously has a receiving position in which it is configured to receive the spinal spacer in the deployed state inside the cavity. The compression device advantageously has a compression position in which it compresses the spinal spacer.

When the compression device is in the receiving position, the transverse dimensions of the cavity, considered transversely to the longitudinal direction of the cavity, are greater than said transverse dimensions when the compression device is in the compression position. In other words, the cross section of the cavity is smaller when the compression device is brought into the compression position.

The cavity is preferably in the form of a tube when the compression device is in the compression position. The diameter of this tube is preferably less than or equal to the diameter of the implantation tube.

The compression device is preferably configured to bring the spinal spacer to the compressed state by reducing the dimensions of the cavity in at least one direction, preferably in a direction transverse to the longitudinal direction of said cavity. The compression device can preferably be used to compress the spinal spacer radially.

When it is in the compressed state, the spinal spacer may be arranged in the implantation tube of the guide device. More precisely, when it is in the compressed state, the transverse dimensions of the spinal spacer, considered transversely to its main direction, are less than the diameter of the implantation tube.

By virtue of the disclosure, the compression device can be used to effectively compress the spinal spacer and easily bring it from the deployed state to the compressed state when it is actuated. The force required to bring the spinal spacer to the compressed state is therefore reduced. The disclosure eliminates the need for a separate tool that has not been adapted, such as pliers, to bring the spinal spacer to the compressed state.

In particular, the compression device can compress the spinal spacer sufficiently to allow it to be inserted into a guide device with a small cross section or a small external diameter, generally of between 12 and 16 millimeters, which can be used to implement a minimally invasive implantation technique. The spinal implant system according to the disclosure is therefore suitable for minimally invasive implantation.

The compression device preferably comprises a press.

The guide device is configured to guide the spinal spacer to between two vertebrae and thus allow it to be implanted. It advantageously has a generally cylindrical shape with a circular cross section. The guide device advantageously has a smaller external diameter than the pipes of the devices of the prior art, thus allowing it to be used to implement a minimally invasive technique. The guide device preferably has an external diameter, at least at its distal end, smaller than 20 millimeters, preferably smaller than 16 millimeters, and more preferably of between 12 and 16 millimeters.

The implantation tube is formed in the guide device. The implantation tube has a circular cross section. The implantation tube advantageously has a constant cross section. The implantation tube is configured to receive the spinal spacer in the compressed state and to guide the translational movement of the spinal spacer up to the distal end of the guide device. In other words, the implantation tube forms a duct for receiving the spinal spacer in the compressed state. The implantation tube advantageously extends along a guide axis.

The distal end of the guide device advantageously has a distal opening. The distal opening is advantageously an axial opening. The distal opening formed in the distal end of the guide device leads into the implantation tube.

The guide device advantageously comprises a proximal end in which a proximal opening is formed for inserting the spinal spacer into the implantation tube. Said proximal opening leads into the implantation tube. The proximal opening is advantageously an axial opening considered along the guide axis of the guide device. The proximal end of the guide device is advantageously configured to be connected to said compression device.

The guide device is preferably configured to be removably connected to the compression device.

The connection between the guide device and the compression device preferably securely connects said guide device and said compression device. In other words, when the guide device is connected to the compression device, said guide device is advantageously secured to the compression device.

The implantation tube preferably defines a channel intended to receive the spinal spacer in the compressed state.

The implantation tube advantageously has a diameter slightly greater than or equal to the transverse dimensions of the spinal spacer in the compressed state, meaning that the spinal spacer is kept in the compressed state when it is arranged in said implantation tube. The implantation tube advantageously has a diameter of between 10 millimeters and 14 millimeters. The diameter of the implantation tube corresponds to an internal diameter of the guide device.

In a non-limiting manner, when the spinal spacer is in the deployed state, it has at least one dimension, preferably a height, that is greater than the diameter of the implantation tube, meaning that it cannot be inserted into the implantation tube.

In a non-limiting manner, the guide device may comprise only one piece, in which case the implantation tube advantageously comprises a single tube portion formed in said one piece.

Alternatively, and without going beyond the ambit of the disclosure, the guide device may comprise a plurality of pieces that can be assembled together, in which case the implantation tube advantageously comprises a plurality of tube portions, each being formed in one of said pieces.

The pusher device of the implant system according to the disclosure is configured to exert a pushing force in a pushing direction on the spinal spacer. It is configured to be brought into cooperation successively with the compression device and the guide device.

Said pushing direction preferably coincides with the longitudinal direction of the cavity of the compression device when the pusher device is cooperating with the compression device. The pushing direction preferably coincides with the guide axis of the implantation tube of the guide device when the pusher device is cooperating with the guide device.

The pusher device is preferably configured to be removably connected to the compression device. The pusher device is preferably configured to be removably connected to the guide device. The connection between the pusher device and the compression device preferably securely connects said pusher device and said compression device.

In a non-limiting manner, the spinal implantation of the spinal spacer by means of the spinal implant system may be carried out as follows.

Firstly, the spinal spacer is positioned in the deployed state in the cavity of the compression device. The compression device is then actuated in such a way as to compress the spinal spacer and bring it into the compressed state. In the compressed state, the spinal spacer has dimensions transverse to its main direction that are smaller than the diameter of the implantation tube and can therefore be inserted into the latter.

The pusher device is placed in cooperation with the compression device. The pushing direction then substantially coincides with the longitudinal direction of the cavity.

The guide device is also connected to the compression device so that the implantation tube extends in the continuation of the cavity of the compression device. The guide device is then attached to the compression device. The longitudinal direction of the cavity of the compression device and the guide axis of the guide device then substantially coincide. The cavity of the compression device then leads into the implantation tube.

When they cooperate with the compression device, the guide device and the pusher device preferably extend to either side of the compression device.

When the guide device and the pusher device cooperate with the compression device, the pushing direction of the pusher device and the guide axis of the implantation tube preferably substantially coincide.

In a non-limiting manner, the compression device can be actuated after bringing the compression device into cooperation with the pusher device and/or with the guide device.

Preferably, when the compression device is actuated, its cavity has a cross section slightly smaller than or substantially equal to the cross section of the implantation tube.

Next, the pusher device is actuated, exerting a pushing force on the spinal spacer in the pushing direction, directed towards the implantation tube of the guide device. The spinal spacer is then moved in translation from the cavity of the compression device into the implantation tube of the guide device. The spinal spacer passes through the proximal opening formed in the proximal end of the guide device. It should be understood that the spinal spacer is conveyed into the implantation tube, and therefore the guide device, close to the proximal end of the guide device.

The spinal spacer is advantageously moved in the longitudinal direction of the cavity.

The spinal spacer is kept in the compressed state as it passes from the cavity of the compression device into the implantation tube.

The pusher device can be used to transfer the spinal spacer from the compression device into the implantation tube. The spinal spacer is kept in the compressed state during this transfer and is not at risk of being deployed accidentally. In other words, the spinal implant system makes it easier to compress the spinal spacer and position it, in the compressed state, inside the implantation tube of the guide device. The disclosure therefore eliminates the need for complex handling operations in order to arrange the spinal spacer in the guide device in the compressed state. This makes it easier to insert the spinal spacer into the guide device and more generally to implant it.

The pusher device is then brought into cooperation with the guide device, and preferably connected to the guide device. Preferably, the pusher device is brought into cooperation with the proximal end of the guide device.

The pusher device is then actuated in such a way as to exert a pushing force on the spinal spacer, which is moved in translation along the implantation tube. The spinal spacer is moved in translation along the guide axis of the implantation tube. The pusher device is configured to move the spinal spacer from the proximal end up to the distal end of the guide device. The spinal spacer is kept in the compressed state throughout its movement through the implantation tube. It is conveyed up to the open distal end of the guide device where it is extracted from the guide device in order to be arranged between two successive vertebrae, where it returns to its deployed state and is implanted.

Moreover, the guide device of the implant system according to the disclosure is used to guide the spinal spacer up to the area to be treated so that the spinal spacer does not damage tissues when it is being implanted.

Advantageously, the guide device has a proximal end, opposite said distal end, configured to be connected to the compression device or to the pusher device. The guide device can therefore be connected alternatively to the pusher device or to the compression device.

One advantage is that the compression device and the guide device can be kept assembled in order to prevent them from being separated from each other. This allows the spinal spacer to be conveyed directly from the compression device into the guide device, without the risk of it being deployed. Another advantage is that the spinal spacer can be conveyed into the guide device only by actuating the pusher device, without the need to hold the pusher device and the compression device manually, and in particular to position them correctly relative to each other when the pusher device is actuated.

Another advantage is that the pusher device and the guide device can be kept assembled. This also allows the spinal spacer to be moved along the implantation tube without the need to manually hold the pusher device and the guide device and position them correctly relative to each other when the pusher device is actuated. This makes it easier to convey the spinal spacer up to the distal end of the guide device.

The guide device preferably comprises a first connector portion, the compression device comprises a second connector portion configured to cooperate with the first connector portion to connect the guide device to the compression device, and the pusher device comprises a third connector portion configured to cooperate with the first connector portion to connect the guide device to the pusher device.

It should be understood that the guide device can easily be connected to or disconnected from the compression device and the pusher device. The first and second connector portions prevent the guide device from moving in translation relative to the compression device. The first and third connector portions prevent the guide device from moving in translation relative to the pusher device.

Advantageously, the first connector portion and at least one of said second and third connector portions comprise complementary bayonet connection portions. “Complementary bayonet connection portions” should be understood to mean at least one female bayonet connection portion and at least one male bayonet connection portion. One advantage is that the guide device can be quickly and easily connected to and/or disconnected from the pusher device or the compression device. A male bayonet connection portion advantageously comprises at least one pin. A female bayonet connection portion advantageously comprises a notch formed in the corresponding device.

Preferably, the first connector portion comprises at least one female bayonet connection portion while the second connector portion and/or the third connector portion comprises at least one male bayonet connection portion. Alternatively, the first connector portion comprises at least one male bayonet connection portion while the second connector portion and/or the third connector portion comprises at least one female bayonet connection portion.

According to another advantageous variant, the first connector portion comprises a flange formed at the proximal end of the guide device or a locking ring situated at the proximal end of the guide device, and at least one of the second and third connector portions comprises a locking ring configured to cooperate with said flange, or a flange configured to cooperate with said locking ring, in order to connect the guide device to the compression device or to the pusher device.

The locking ring advantageously has a pivoting ring portion that can be opened or closed. Dans this closed position, the locking ring is configured to clamp the flange to prevent relative translational movement of the two connector portions.

Said guide device preferably comprises:
a transfer cartridge comprising a first tube portion and being configured to be connected to the compression device; and
a guide pipe configured to be connected to said transfer cartridge and comprising a second tube portion, said first and second tube portions defining said implantation tube,
   the pusher device being configured to cooperate:
with the compression device, when the transfer cartridge is connected thereto, to push the spinal spacer in the compressed state from the cavity of the compression device into the first tube portion of the transfer cartridge; and
with said transfer cartridge, when the guide pipe is connected thereto, to push the spinal spacer in the compressed state from the inside of the first tube portion up to the distal end of the guide device via the inside of the second tube portion, so as to extract said spinal spacer out of the guide device.

In this non-limiting embodiment, the guide device comprises two separate pieces. The transfer cartridge is advantageously in the form of a cylinder with a circular cross section. The guide pipe is advantageously cylindrical in shape, and even more preferably circular in cross-section. The first tube portion advantageously extends along a cartridge axis. The second tube portion advantageously extends along a pipe axis.

The transfer cartridge preferably has a first end configured to be connected to the compression device and/or to the pusher device. This first end corresponds to the proximal end of the guide device. The transfer cartridge preferably has a second end, opposite the first end, configured to be connected to the guide pipe.

The guide pipe preferably has a first end part configured to be connected to the transfer cartridge and a second end part opposite the first end part. This second end part corresponds to the distal end of the guide device.

When the transfer cartridge is connected to the compression device, said first tube portion extends in the continuation of the cavity of the compression device. Furthermore, the cartridge axis then advantageously coincides with the longitudinal direction of the cavity. When the guide pipe is connected to the transfer cartridge, said second tube portion extends in the continuation of the first tube portion. Furthermore, the pipe axis and the cartridge axis then advantageously coincide. Moreover, the pipe axis and the cartridge axis then advantageously coincide with the guide axis of the implantation tube of the guide device.

The second tube portion preferably has a diameter that is substantially equal, and is preferably equal, to the diameter of the first tube portion.

The guide pipe allows the spinal spacer to be implanted and its second end part is intended to be positioned close to the vertebrae of the patient.

In order to implant the spinal spacer, the transfer cartridge is firstly connected to the compression device and the pusher device is actuated to push the spinal spacer into the first tube portion, and therefore into the transfer cartridge.

The transfer cartridge is then disconnected from the compression device and connected to the guide pipe and brought into cooperation with the pusher device. The pusher device can then be used to move the spinal spacer from the inside of the first tube portion of the transfer cartridge into the second tube portion of the guide pipe, then up to the distal end of the guide device.

In this configuration, the transfer cartridge forms a buffer receptacle intended to temporarily receive the spinal spacer in the compressed state before it is positioned in the guide pipe. Using a transfer cartridge eliminates the need to connect the guide pipe to the compression device when transferring the spinal spacer between the compression device and the guide device. The guide pipe may be kept close to the implantation area and the risk of contaminating the guide pipe is therefore reduced.

Alternatively, without going beyond the ambit of the invention, the guide pipe may be kept connected to the transfer cartridge when the spinal spacer is conveyed from the cavity of the compression device into said transfer cartridge.

The transfer cartridge is preferably configured to be connected to the pusher device.

The length of said transfer cartridge is preferably less than half of the length of the guide pipe. The length of the transfer cartridge is preferably slightly greater than or substantially equal to the length of a spinal spacer to be implanted. One advantage is that the space requirement of the guide device is reduced.

Advantageously, said guide pipe is configured to be connected to said transfer cartridge by means of a bayonet connection or by means of a clamping ring configured to cooperate with a flange. One advantage is that the guide pipe and the transfer cartridge can be connected and disconnected easily and quickly.

The transfer cartridge preferably comprises at least one female bayonet connection portion and the guide pipe comprises at least one male bayonet connection portion, for example a pin extending radially into the guide pipe.

Advantageously, said guide pipe comprises a tubular grip part and a tubular body connected to said tubular grip part, said tubular grip part having an external diameter greater than an external diameter of the tubular body. One advantage is that the handling of the guide pipe and more generally of the guide device is facilitated. Said grip portion advantageously extends between the transfer cartridge and the tubular body.

The compression device preferably comprises a bottom jaw, a top jaw and a clamping member configured to move said top and bottom jaws towards or away from each other. Actuating the clamping member moves the top and bottom jaws towards or away from each other. Moving the jaws towards each other compresses the spinal spacer positioned in the cavity. The cavity is formed between the two jaws. Such a compression device helps effectively compress the spinal spacer while reducing the force that needs to be applied by the operator.

Preferably, only one of the two jaws is able to move in translation, while the other jaw is fixed. More preferably still, only the top jaw is above to move. The clamping member advantageously allows the top jaw to be moved relative to the bottom jaw. The top jaw is preferably able to move in translation relative to the bottom jaw. The clamping member advantageously allows the top jaw to be moved along a clamping axis.

When the compression device is in the compression position, the top and bottom jaws are advantageously touching.

The clamping member advantageously comprises a threaded rod and a clamping wheel.

The cavity of the compression device is preferably cylindrical. The compression device is advantageously capable of assuming a compression position in which the cavity is cylindrical with a circular cross section.

Preferably, a first semi-cylindrical groove is formed in the bottom jaw, a second semi-cylindrical groove is formed in the top jaw, said first and second semi-cylindrical grooves together defining said cavity of said compression device. One advantage is that the spinal spacer is effectively kept in the compressed state in the cavity, preventing it from being able to exit the cavity of the compression device radially.

The pusher device preferably comprises a main body, a piston and a trigger configured to drive the translational movement of the piston in relation to the main body when it is actuated.

The pusher device is advantageously in the form of a gun, such as a caulking gun.

The piston is configured to exert a pushing force on the spinal spacer in the compressed state. The piston advantageously extends in the pushing direction of the pusher device.

Advantageously, the piston comprises a rod and a piston head, the diameter of said piston head being smaller than the diameter of the implantation tube of the guide device. One advantage is that the piston can enter the implantation tube of the guide device so as to be able to effectively push the spinal spacer up to the distal end of the guide device.

The length of the rod of the piston is preferably greater than or equal to the length of the guide device.

The diameter of the piston head is preferably smaller than the diameter of the cavity of the compression device when the latter is in the compression position. One advantage is that the piston can enter the cavity.

Advantageously, the distal end of the guide device is at least partially beveled. One advantage is that the positioning of the distal end of the guide device as close as possible to the area where the spinal spacer is to be implanted is facilitated.

The distal end of the guide device advantageously comprises a domed engagement portion. This engagement portion is configured to come to bear on one of the vertebrae in the implantation area and prevents damage to tissues, ligaments and bones.

The engagement portion is preferably in the form of a quarter of a sphere.

### BRIEF DESCRIPTION OF THE DRAWINGS

The invention will be more clearly understood upon reading the description that follows of an embodiment of the invention provided as a non-limiting example, in reference to the appended drawings, in which:

FIG. 1 shows a guide device of a spinal implant system according to the disclosure;

FIG. 2 shows the transfer cartridge of the guide device of FIG. 1;

FIG. 3 is a first perspective view of the guide pipe of the guide device of FIG. 1;

FIG. 4 is a second perspective view of the guide pipe of the guide device of FIG. 1;

FIG. 5 shows the compression device of the spinal implant system according to the disclosure;

FIG. 6 shows the pusher device of the spinal implant system according to the disclosure;

FIG. 7 shows a spinal spacer being inserted into the cavity of the compression device of FIG. 5;

FIG. 8 shows the spinal spacer being compressed by means of the compression device of FIG. 5;

FIG. 9 shows the spinal spacer pushed into the transfer cartridge by means of the pusher device of FIG. 6;

FIG. 10 shows the spinal spacer in the compressed state arranged in the transfer cartridge;

FIG. 11 shows the spinal spacer pushed along the implantation tube;

FIG. 12 shows the spinal spacer being extracted from the guide device; and

FIG. 13 shows the spinal spacer being implanted between two vertebrae by means of the spinal implant system according to the disclosure.

### DESCRIPTION OF EMBODIMENTS

The invention relates to a spinal implant system for a spinal spacer allowing such a spinal spacer to be implanted, in particular between two successive vertebrae.

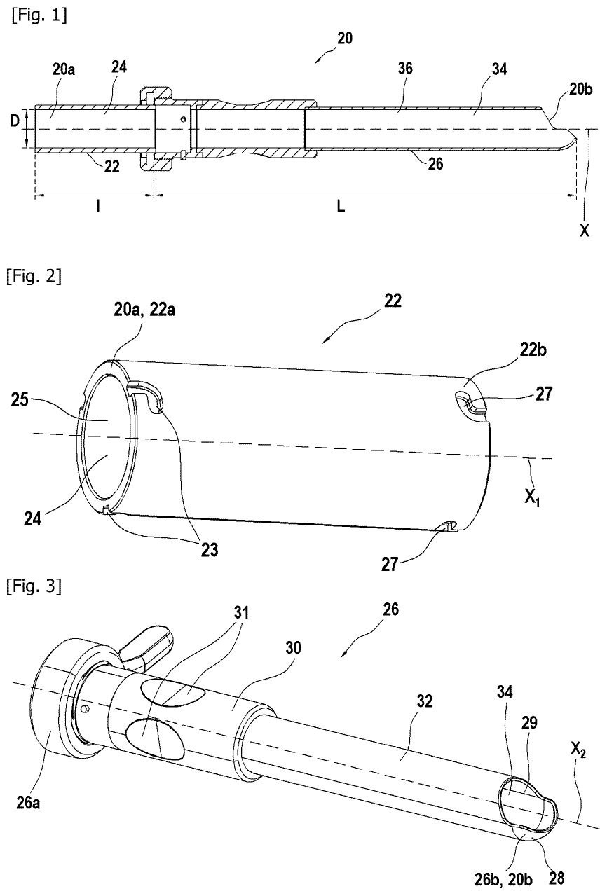

A non-limiting embodiment of such a spinal implant system 10 according to the disclosure will be described in reference to FIGS. 1 to 13.

According to the disclosure, the spinal implant system 10 comprises a guide device 20, a compression device 40 and a pusher device 60.

FIG. 1 shows a guide device 20 of the spinal implant system 10 according to the disclosure. The guide device 20 has a generally cylindrical shape, with a circular cross section. It has an open proximal end 20a and a distal end 20b that is also open. The distal end 20b of the guide device is beveled. The guide device 20 extends along a guide axis X.

In this non-limiting example, the guide device 20 comprises a tubular transfer cartridge 22 shown in FIG. 2. The transfer cartridge 22 has a first end 22a, constituting said proximal end 20a of the guide device 20, and a second end 22b. The transfer cartridge 22 comprises a first tube portion 24. The first tube portion 24 forms a duct inside the transfer cartridge 22. A proximal opening 25 is provided axially in said transfer cartridge 22 and leads into said first tube portion 24. The transfer cartridge 22 and the first tube portion 24 extend along a cartridge axis X₁.

The guide device 20 and, more precisely, the first end 22a of the transfer cartridge 22, comprises three female bayonet connection portions together forming a first connector portion 23 for the guide device. The second end 22b of the transfer cartridge 22 also comprises three female bayonet connection portions 27.

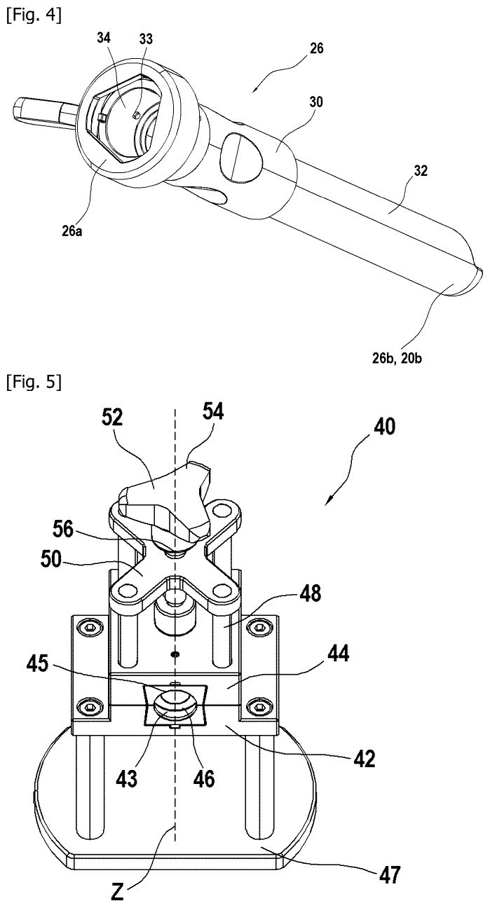

The guide device 20 further comprises a guide pipe 26 of generally cylindrical shape, with a circular cross section, shown in FIGS. 3 and 4. The guide pipe 26 extends along a pipe axis X₂. The guide pipe 26 has a first end part 26a and a second end part 26b, corresponding to the distal end 20b of the guide device 20.

As shown in FIG. 1, the length I of said transfer cartridge 22 is less than half the length L of the guide pipe 26.

A distal opening 29 is provided axially in the second end part 26b of the guide pipe 26. Moreover, as can be seen in FIG. 3, the second end part 26b of the guide pipe 26, and therefore the distal end 20b of the guide device 20, comprise a domed engagement portion 28.

The guide pipe 26 comprises a tubular grip part 30 and a tubular body 32 linked to said tubular grip part 30 and extending in the continuation of the tubular grip part. The tubular body 32 and said tubular grip part 30 extend along the pipe axis X₂. The tubular grip part 30 has an external diameter greater than an external diameter of the tubular body 32. The tubular grip part 30 comprises indentations 31 that make it easier to hold the guide device.

The guide pipe 26 comprises a second tube portion 34 extending along the pipe axis X₂. The second tube portion 34 forms a duct inside the guide pipe. The diameter of the second tube portion 34 is substantially equal to the diameter of the first tube portion 24. The diameter of the second tube portion 34 corresponds to the internal diameter of the guide pipe 26.

In reference to FIG. 4, it can be seen that the first end part 26a of the guide pipe 26 comprises three connection pins 33 extending radially towards the inside of the guide pipe 26. These connection pins 33 form male bayonet connection portions. The connection pins 33 are configured to engage with the female bayonet connection portions 27 of the second end 22b of the transfer cartridge 22, in order to removably connect the guide pipe 26 to the transfer cartridge 22.

Referring once more to FIG. 1, it can be seen that, when the transfer cartridge 22 is connected to the guide pipe 26, the cartridge axis X₁ and the pipe axis X₂ coincide with each other and with the guide axis X of the guide device. The guide device 20 comprises an implantation tube 36 defined by the first and second tube portions 24,34. In this non-limiting example, said first and second tube portions 24,34 form the implantation tube 36. The implantation tube 36 defines a duct inside the guide device 20. The implantation tube extends along the guide axis X. The implantation tube 36 has a diameter constituting an internal diameter for the guide device 20.

FIG. 5 shows a compression device 40 of the spinal implant system 10. The compression device 40 comprises a press. The compression device 40 comprises a bottom jaw 42 and a top jaw 44 arranged above the bottom jaw 42.

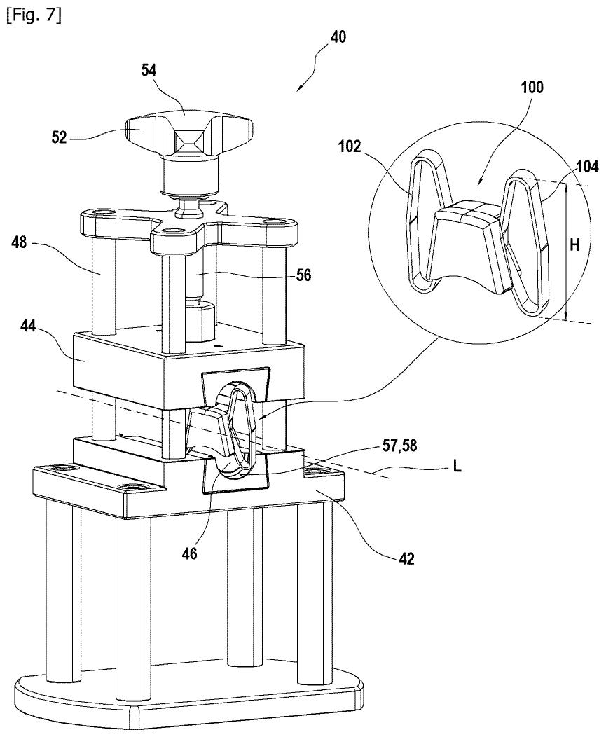

A first semi-cylindrical groove 43 is formed in the bottom jaw 42 whereas a second semi-cylindrical groove 45 is formed in the top jaw 44. The first and second semi-cylindrical grooves together form a cylindrical cavity 46. The cavity 46 extends in a longitudinal direction L_{d} shown in FIG. 7.

The bottom jaw 42 is mounted on a support 47. The compression device 40 comprises four guide rods 48 fastened to the bottom jaw 42 and protruding transversely to said bottom jaw, vertically. The guide rods 48 extend parallel to each other. A mounting piece 50 is fastened to the top ends of the guide rods 48. The top jaw 44 is mounted so as to be able to move in translation along the guide rods 48 according to a clamping axis Z which is in this case vertical. It is also prevented from rotating.

The compression device 40 further comprises a clamping member 52 configured to move the top jaw 44 in relation to the bottom jaw 42 along said clamping axis Z. The clamping member 52 extends along the clamping axis Z. In this non-limiting example, the clamping member 52 comprises a clamping wheel 54 connected to a threaded rod 56 cooperating with a tapped hole formed in the top jaw 44.

The clamping member is prevented from moving in translation in relation to the mounting piece 50 and is in helical sliding connection with the top jaw 44. When the clamping member 52 is pivoted about its axis, it moves the top jaw 44 in translation along the guide rods 48. By actuating the clamping member 52, the top jaw can therefore be moved towards the bottom jaw 42 in order to reduce the size of the cavity 46. When the top and bottom jaws 44, 42 are touching, the cavity has a circular cylindrical shape. The top jaw 44 can also be moved away from the bottom jaw 42 in such a way as to increase the size of the cavity 46.

As shown in FIG. 7, the compression device 40 also comprises three connection pins 57 that together form a second connector portion 58 of the male bayonet connector type. These connection pins 57 of the second connector portion 58 are configured to cooperate with the female bayonet connection portions of the first connector portion 23 of the guide device 20 in order to removably connect the guide device to the compression device 40.

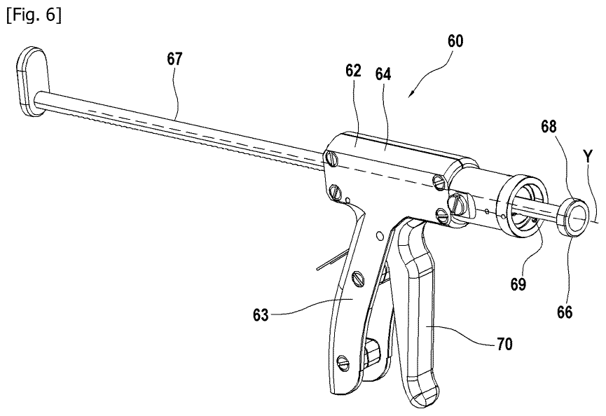

As shown in FIG. 6, the spinal implant system 10 further comprises a pusher device 60 designed in the form of a caulking gun. The pusher device 60 comprises a main body 62 comprising a handle 63 and a cylindrical portion 64 extending in a pushing direction Y. The pusher device 60 further comprises a piston 66 mounted so as to be able to move in translation relative to the main body 62 in the pushing direction Y. More precisely, the piston 66 comprises a piston rod 67 and a piston head 68 fastened to the end of the piston rod 67. The piston rod 67 is mounted so as to be able to move in translation inside the cylindrical portion 64 of the main body 62, in the pushing direction Y. The piston rod 67 passes through said cylindrical portion 64. The pusher device 60 further comprises a trigger 70 mounted so as to be able to pivot relative to the main body 62. The trigger is configured to drive the translational movement of the piston 66 relative to the main body, in the pushing direction Y, when it is actuated.

The cylindrical portion 64 of the main body 62 of the pusher device 60 further comprises three connection pins 69 protruding radially into said cylindrical portion. The connection pins 69 form a third connector portion 69. These connection pins constitute male bayonet connection portions.

The implantation of a spinal spacer 100 by means of the spinal implant system 10 will now be described in detail in reference to FIGS. 7 to 11.

Firstly, a spinal spacer 100 is provided that is elastically deformable between a deployed state and a compressed state. In this non-limiting example, a spinal spacer 100 such as that described in document EP 4 149 376 A1 is provided. The spinal spacer 100 in this case comprises two elastically deformable wings 102,104 that can be compressed in order to reduce the height H thereof. The spinal spacer extends in a main direction.

As shown in FIG. 7, the compression device 40 is first placed in a receiving position in which its top and bottom jaws 44,42 are spaced apart from each other. The size of the cavity 46 is then sufficient to receive the spinal spacer 100 in the deployed state.

The spinal spacer 100 is then inserted into the cavity 46 of the compression device 40, between the bottom jaw 42 and the top jaw 44. The spinal spacer 100 is in the deployed state, forming a resting state in which the height H of its wings 102,104 is at a maximum. In this deployed state, the spinal spacer 100 cannot be inserted into the implantation tube 36 of the guide device 20, insofar as the height H of its wings is greater than the diameter of the implantation tube 36.

The clamping member 52 is then actuated by pivoting the clamping wheel 54 in a clockwise direction. As a result, the top jaw 44 is moved in translation along the guide rods 48 towards the bottom jaw 42. The size of the cavity 46 is reduced and the jaws exert a compression force on the spinal spacer 100 and in particular on the wings 102,104, which has the effect of reducing the height H of the wings and therefore, more generally, of the spinal spacer 100.

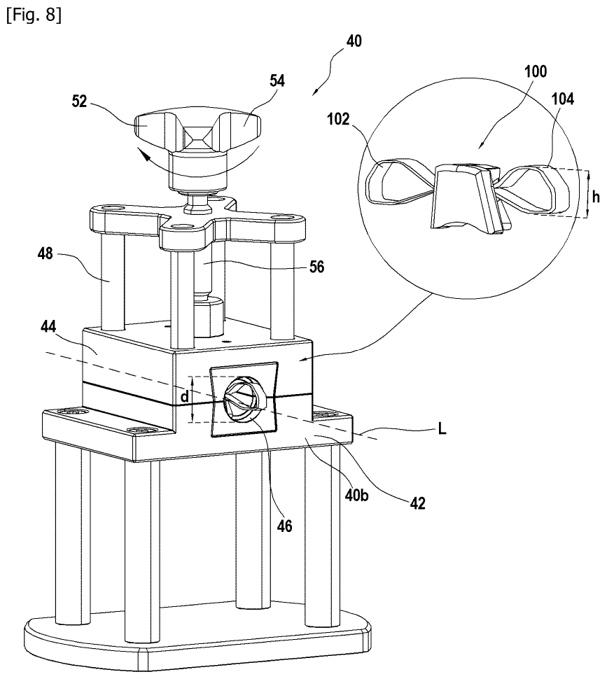

As shown in FIG. 8, when the top and bottom jaws 44,42 are moved as close together as possible and touch, the size of the cavity 46 is reduced to a minimum and the compression device 40 is then in a compression position. The cavity 46 then has a tubular shape and has a diameter d slightly smaller than or substantially equal to the diameter D of the implantation tube 36. Given the compression force exerted by the compression device, the spinal spacer 100 is then placed in the compressed state and the height h of its wings is reduced. The height of the wings of the spinal spacer 100 and of the spinal spacer more generally is then less than the diameter of the implantation tube 36.

The compression device 40 of the spinal implant system 10 therefore makes it possible to reduce the transverse dimensions and, in particular, the height of the spinal spacer 100 in order to allow it to be inserted into the guide device 20. This compression device 40 also makes it possible to significantly compress the spinal spacer in order to be able to arrange it in an implantation tube with a small diameter, making it possible to use a guide device 20 with small internal and external diameters that is compatible with a minimally invasive spinal implantation technique (MISS).

The diameter of the cavity 46 when the compression device 40 is in this compression position is slightly greater than the diameter of the piston head 68 of the piston 66 of the pusher device 60.

The top jaw 44 and bottom jaw 42 then define a first face 40a and a second face 40b, opposite the first face, for the compression device 40.

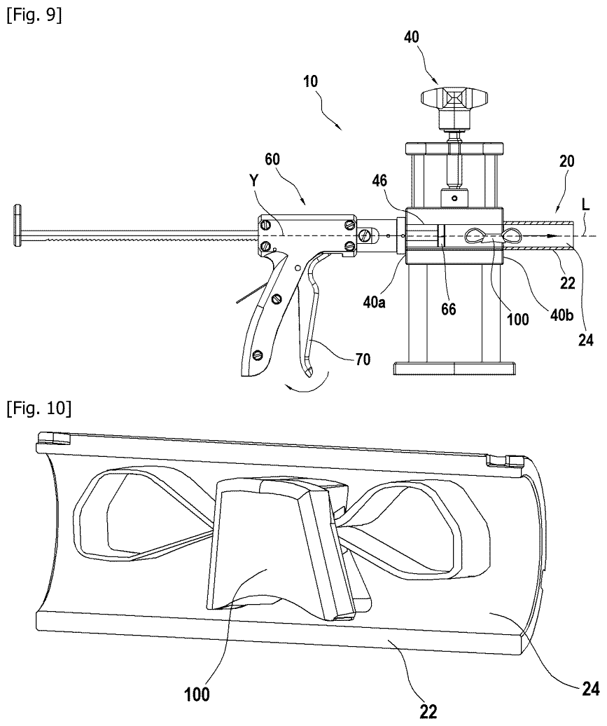

As shown in FIG. 9, the pusher device 60 is then brought into cooperation with the compression device 40. The piston 66 is positioned in line with the cavity 46 and the pusher device 60 is positioned so that the pushing direction Y substantially coincides with the longitudinal direction L_{d} of the cavity 46. The pusher device 60 is positioned such that the cylindrical portion 64 of the main body 62 comes into contact with the first face 40a of the compression device.

Moreover, the first connector portion 23 of the guide device 20 and the transfer cartridge 22 is brought into cooperation with the second connector portion 58 of the compression device 40. Said transfer cartridge 22 is thus connected to the compression device 40. The first tube portion 24 formed in the transfer cartridge 22 then extends in the continuation of the cavity 46. Furthermore, the cartridge axis X₁ then substantially coincides with the longitudinal direction L_{d} of the cavity 46.

The pusher device 60 is then actuated by pivoting the trigger 70. The piston 66 is moved in translation in the pushing direction Y and pushes the spinal spacer 100 in said pushing direction. Insofar as the diameter of the piston head is smaller than the diameter of the cavity 46, the piston enters the cavity 46 at the same time. Given the pushing force exerted by the piston 66, the spinal spacer 100 is moved into the first tube portion 24, and therefore into the implantation tube 36 and the guide device 20. The spinal spacer 100 passes directly from inside the cavity 46 into the first tube portion 24 of the transfer cartridge 22, and is kept in the compressed state.

It is easier to insert the spinal spacer into the guide device 20, and the spinal spacer 100 is not at risk of being deployed when it is conveyed into the guide device.

The spinal spacer 100 is conveyed completely into the transfer cartridge 22, in the first tube portion 24, as shown in FIG. 10. The transfer cartridge may then be disconnected from the compression device, the spinal spacer 100 being kept inside said transfer cartridge 22 in the compressed state.

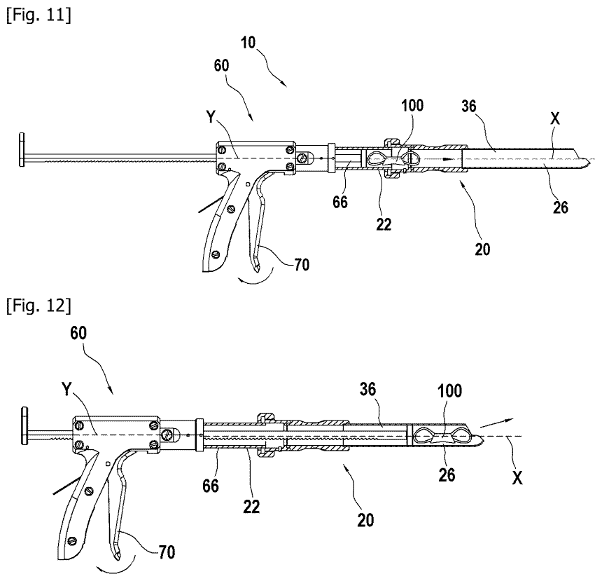

As shown in FIG. 11, the guide pipe 26 and the pusher device 60 are then connected to the transfer cartridge 22.

The transfer cartridge 22 is connected to the guide pipe 26 by means of bayonet connection portions. The guide pipe 26 and the transfer cartridge 22 then extend along the guide axis X of the guide device 20, in the continuation of one another. The transfer cartridge 22 and the guide pipe 26 are thus securely connected. The first and second tube portions 24,34 then together define the implantation tube 36.

Moreover, the pusher device 60 is brought into cooperation with the transfer cartridge 22. More precisely, the pusher device 60 is removably connected to the transfer cartridge 22. For this purpose, the connection pins 69 of the cylindrical portion 64 of the pusher device 60 are brought into cooperation with the female bayonet connection portions 23 of the transfer cartridge 22. In other words, the third connector portion 69 is brought into cooperation with the first connector portion 23.

The pushing direction Y therefore substantially coincides with the guide axis X of the guide device 20.

The pusher device 60 is then actuated, meaning that the piston 66 pushes the spinal spacer 100, in the compressed state, along the implantation tube 36 and therefore along the guide device 20, along the guide axis X. The spinal spacer 100 is moved from the proximal end 20a up to the distal end 20b of the guide device 20. The spinal spacer passes from inside the transfer cartridge 22 into the guide pipe 26, and therefore from the first tube portion 24 to the second tube portion 34.

The diameter of the piston head 68 is smaller than the diameter of the implantation tube 36, meaning that the piston head moves in translation into the implantation tube 36.

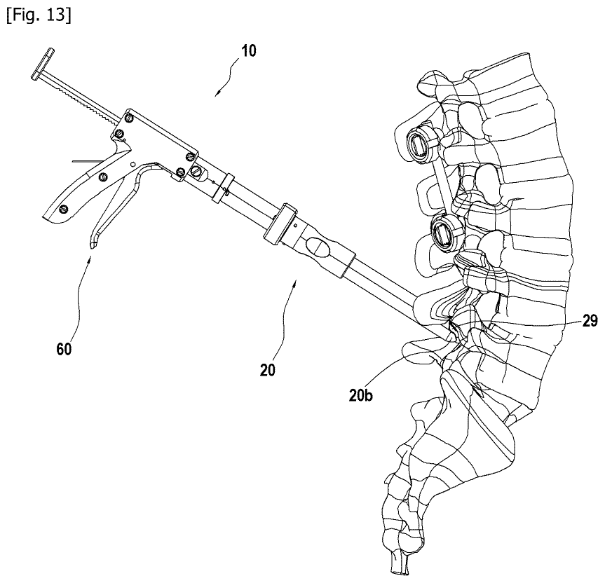

The pusher device 60 is actuated in such a way as to push the spinal spacer 100 in the compressed state up to the distal end 20b of the guide device 20, in order to extract it from the guide device 20, as shown in FIG. 12. The spinal spacer is pushed out through the distal opening 29 of the guide device. The distal end of the guide device 20 is positioned in such a way as to guide the spinal spacer 100 to between two successive vertebrae, where it is deployed and held in position in particular by the wings 102,104, as shown in FIG. 13.

1. A spinal implant system for a spinal spacer that is at least partially elastically deformable between a deployed state and a compressed state, the spinal implant system comprising:
a compression device having a cavity intended to receive the spinal spacer in the deployed state, the compression device being configured to exert a compression force on said spinal spacer situated in said cavity so as to cause said spinal spacer to switch from the deployed state to the compressed state;
a guide device having an open distal end and comprising an implantation tube, the guide device being able to be connected to said compression device; and
a pusher device configured to cooperate:
with the compression device, when the guide device is connected thereto, to push the spinal spacer in the compressed state from the cavity of the compression device into the implantation tube of the guide device; and
with the guide device, to push the spinal spacer in the compressed state along the implantation tube up to the distal end of the guide device, so as to extract said spinal spacer out of the guide device.

2. The spinal implant system as claimed in claim 1, wherein the guide device has a proximal end, opposite said distal end, configured to be connected to the compression device or to the pusher device.

3. The spinal implant system as claimed in claim 2, wherein the guide device comprises a first connector portion, wherein the compression device comprises a second connector portion configured to cooperate with the first connector portion to connect the guide device to the compression device, and wherein the pusher device comprises a third connector portion configured to cooperate with the first connector portion to connect the guide device to the pusher device.

4. The spinal implant system as claimed in claim 3, wherein the first connector portion and at least one of said second and third connector portions comprise complementary bayonet connection portions.

5. The spinal implant system as claimed in claim 1, wherein said guide device comprises:
a transfer cartridge comprising a first tube portion and being configured to be connected to the compression device; and
a guide pipe configured to be connected to said transfer cartridge and comprising a second tube portion, said first and second tube portions defining said implantation tube, the pusher device being configured to cooperate:
with the compression device, when the transfer cartridge is connected thereto, to push the spinal spacer in the compressed state from the cavity of the compression device into the first tube portion of the transfer cartridge; and
with said transfer cartridge, when the guide pipe is connected thereto, to push the spinal spacer in the compressed state from the inside of the first tube portion up to the distal end of the guide device via the inside of the second tube portion, so as to extract said spinal spacer out of the guide device.

6. The spinal implant system as claimed in claim 5, wherein the length of said transfer cartridge is less than half the length of the guide pipe.

7. The spinal implant system as claimed in claim 5, wherein said guide pipe is configured to be connected to said transfer cartridge by means of a bayonet connection or by means of a clamping ring configured to cooperate with a flange.

8. The spinal implant system as claimed in claim 5, wherein said guide pipe comprises a tubular grip part and a tubular body connected to said tubular grip part, said tubular grip part having an external diameter greater than an external diameter of the tubular body.

9. The spinal implant system as claimed in claim 1, wherein the compression device comprises a bottom jaw, a top jaw and a clamping member configured to move said top and bottom jaws towards or away from each other.

10. The spinal implant system as claimed in claim 1, wherein the cavity of the compression device is cylindrical.

11. The spinal implant system as claimed in claim 1, wherein the cavity of the compression device is cylindrical and wherein a first semi-cylindrical groove is formed in the bottom jaw, wherein a second semi-cylindrical groove is formed in the top jaw, said first and second semi-cylindrical grooves together defining said cavity of said compression device.

12. The spinal implant system as claimed in claim 1, wherein the pusher device comprises a main body, a piston and a trigger configured to drive the translational movement of the piston in relation to the main body when it is actuated.

13. The spinal implant system as claimed in claim 12, wherein the piston comprises a rod and a piston head, the diameter of said piston head being smaller than the diameter of the implantation tube of the guide device.

14. The spinal implant system as claimed in claim 1, wherein the distal end of the guide device is at least partially beveled.

15. The spinal implant system as claimed in claim 1, wherein the distal end of the guide device comprises a domed engagement portion.

16. A spinal implant system for a spinal spacer that is at least partially elastically deformable between a deployed state and a compressed state, the spinal implant system comprising:
a compression device having a cavity intended to receive the spinal spacer in the deployed state, the compression device being configured to exert a compression force on said spinal spacer situated in said cavity so as to cause said spinal spacer to switch from the deployed state to the compressed state.

17. The spinal implant system of claim 16, further comprising:
a guide device having an open distal end and comprising an implantation tube, the guide device being able to be connected to said compression device.

18. The spinal implant system of claim 17, further comprising:
a pusher device configured to cooperate:
with the compression device, when the guide device is connected thereto, to push the spinal spacer in the compressed state from the cavity of the compression device into the implantation tube of the guide device; and
with the guide device, to push the spinal spacer in the compressed state along the implantation tube up to the distal end of the guide device, so as to extract said spinal spacer out of the guide device.
